(19) United States
(12) Patent Application Publication
(10) Pub. No.: US 2024/0237902 A1
(43) Pub. Date: Jul. 18, 2024

(54) SYSTEMS AND METHODS FOR DIAGNOSING PERIPHERAL ARTERIAL DISEASE (PAD) USING GAIT ACCELERATION CHARACTERISTICS

(71) Applicant: NUtech Ventures, Lincoln, NE (US)

(72) Inventors: Fadi Alsaleem, Lincoln, NE (US); Mohammad A. Takallou, Lincoln, NE (US); Sara Myers, Lincoln, NE (US); Iraklis Pipinos, Lincoln, NE (US); Basheer Qolomany, Lincoln, NE (US)

(21) Appl. No.: 18/392,260

(22) Filed: Dec. 21, 2023

Related U.S. Application Data
(60) Provisional application No. 63/476,862, filed on Dec. 22, 2022.

(51) Int. Cl. A61B 5/02; A61B 5/00; A61B 5/11; G06N 20/00

(52) U.S. Cl. CPC A61B 5/02007; A61B 5/112; A61B 5/6802; G06N 20/00

(57) ABSTRACT

Systems and methods for diagnosing peripheral artery disease (PAD) in a patient. The method includes training a machine learning model with gait characteristic data extracted from acceleration data for patients known to have PAD and patients that do not have PAD, receiving acceleration data related to a specific patient, extracting gait characteristic data from the acceleration data related to the specific patient, feeding the extracted gait characteristic data to the trained machine learning model to identify one or more gait features for the specific patient, and diagnosing the specific patient as having PAD or not having PAD based on the one or more identified gait features. The machine learning model can also be trained using biomechanics data extracted using a digital camera, e.g., a high-speed digital camera motion capture system.

### CROSS-REFERENCES TO RELATED APPLICATIONS

This application claims the benefit of U.S. Provisional Application No. 63/476,862 filed on Dec. 22, 2022, the disclosure of which is hereby incorporated by reference.

### BACKGROUND

The present disclosure relates to systems and methods for diagnosing patients with peripheral artery disease (PAD). PAD is an atherosclerotic syndrome that is caused by occlusion of the arteries supplying the legs. While PAD affects approximately 10% of the American population over 40 years of age, 40% to 60% of patients with PAD go undiagnosed in a primary care setting. Diagnosis of PAD primarily occurs using the ankle-brachial index (ABI), a high-cost, specialized test performed in a vascular lab. Recent research has implemented a data-driven approach using machine learning for the identification of patients with PAD. Diagnostic machine learning models used protein biomarkers obtained from blood samples, clinical data extracted through extensive participant interviews and patient history and medical records, doppler images combined with medical records, and arterial pulse waveforms. While a few of these approaches have achieved accuracy up to 87%, they have significant limitations in terms of time (require few years of medical records), resources (require protein-based lab-setting), and involvement of experts with advanced training (physicians, trained research assistants, and nurse practitioners). Functional impairment usually occurs well before PAD is diagnosed and unidentified, asymptomatic PAD is associated with more adverse outcomes than identified symptomatic PAD presenting with intermittent claudication.

Accordingly there is a need for improved systems and methods for diagnosing PAD.

### SUMMARY

The present disclosure provides systems and methods for diagnosing patients with peripheral artery disease (PAD) by analyzing their body acceleration measurements when they walk for only a short period of time, e.g., a few minutes. According to the present embodiments, functional impairments due to PAD can be detected using machine learning methods and acceleration measurements of a patient with PAD. The systems and methods according to the present embodiments use acceleration measurements, e.g., from wearable devices worn by patients, in concert with machine learning to improve the diagnosis and treatment of functional problems in patients with PAD outside the clinical setting. Collected laboratory-based biomechanics and acceleration measurements of patients with PAD are used to develop and train machine learning models to identify unique gait features.

According to an embodiment, a method, e.g., a computer-implemented method, of diagnosing peripheral artery disease (PAD) in a patient is provided. The method includes training a machine learning model with biometric data and gait characteristic data extracted from acceleration data for patients known to have PAD and patients that do not have PAD. The method further includes receiving acceleration data related to a specific patient, extracting gait characteristic data from the acceleration data related to the specific patient, and feeding the extracted gait characteristic data to the trained machine learning model to enable the trained machine learning model to identify one or more gait features for the specific patient. The method may also include diagnosing the specific patient as having PAD or not having PAD based on the one or more identified gait features.

According to an embodiment, a system for diagnosing peripheral artery disease (PAD) in a patient is provided. The system includes a processor and a memory storing instructions, which when executed by the processor causes the processor to access or execute a trained a machine learning model, the trained machine learning model having been trained with biometric data and gait characteristic data extracted from acceleration data for patients known to have PAD and patients that do not have PAD. The system also includes one or more sensors configured to be worn by a specific patient. In operation, the processor is configured to receive acceleration data related to the specific patient generated by the one or more sensors, extract gait characteristic data from the acceleration data related to the specific patient, and feed the extracted gait characteristic data to the trained machine learning model to enable the model to identify one or more gait features for the specific patient. The processor may further be configured to diagnose the specific patient as having PAD or not having PAD based on the one or more identified gait features.

According to an embodiment, a computer-implemented method of diagnosing peripheral artery disease (PAD) in a patient is provided that includes training a machine learning model with acceleration or accelerometer data for patients known to have PAD and patients that do not have PAD, receiving acceleration data related to a specific patient, feeding the acceleration data to the trained machine learning model to identify one or more gait features for the specific patient, and diagnosing the specific patient as having PAD or not having PAD based on the one or more identified gait features. In certain aspects, the machine learning model comprises a recurrent neural network or a long short-term memory (LSTM) model.

According to another embodiment, a non-transitory computer-readable medium is provided that stores instructions, which when executed by one or more processors, cause the one or more processors to implement any method of training and/or diagnosing peripheral artery disease (PAD) in a patient as disclosed herein.

In certain aspects, the machine learning model comprises one of a neural network algorithm, a recurrent neural network (RNN), a random forest algorithm, a support vector machine (SVM) algorithm and a Logit algorithm. In certain aspects, the acceleration data related to the specific patient is obtained from one or more sensors worn by the specific patient. In certain aspects, the measured acceleration can be used directly to develop a model to diagnose PAD. In certain aspects, one or more gait features include that can be extracted from acceleration measurements such as one or more of step time asymmetry, step time variability, step time, stance time, stride time, and swing time. In certain aspects, the gait characteristic data includes peak discrete point and range of values for one or more of vertical acceleration, anterior acceleration, a number of steps, and a period of time between steps. In certain aspects, the model can be trained by biomechanics data which may include braking impulse, braking peak, propulsive peak, propulsive impulse, and other forces derived from GRF data. In certain aspects, gait characteristic data may include joint torques and powers, such as hip, knee and/or ankle angles, torques and powers. In certain aspects, feeding the data further includes feeding biometric data of the specific patient to the trained machine learning model.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates a system, top, including only acceleration data fed to a RNN learning model, and a system, bottom, including biomechanics data and/or acceleration data fed to a learning model according to embodiments.

FIG. 2 shows a graphic demonstration of gait cycles derived from the vertical dimension of acceleration obtained from the sacral position data, according to an embodiment.

FIG. 3 is a flowchart showing two methods useful for PAD diagnostics according to an embodiment.

FIG. 4 shows a correlation between some of the accelerometer axis measurements.

FIG. 5 shows the RF model's SHAP summary plot generated after being fitted to all features, according to an embodiment.

FIG. 6 shows a schematic comparison of the performance of ML and LSTM models according to embodiments.

FIG. 7 is a schematic showing the advantages of the SVM and LSTM models, according to an embodiment.

### DETAILED DESCRIPTION

The present disclosure provides systems and methods for diagnosing patients with peripheral artery disease (PAD) by analyzing their body acceleration and/or gait kinematic and kinetic measurements using trained machine learning models.

Biomechanics gait analysis has proven its importance for determining the mechanisms and severity of functional limitations, measurement of treatment effectiveness, and monitoring the progression of chronic PAD. Biomechanics gait measurements, while accurate, require extensive laboratory-based testing and limit the number of time points that can be evaluated. Using wearable sensor technology in concert with machine learning algorithms enables extraction of similar measurements at more time points and outside of the clinical setting. The present embodiments integrate information from wearable sensors in a useful way to advantageously develop meaningful clinical applications for machine learning using wearable sensors data.

The present inventors have used advanced biomechanics testing and wearable accelerometers to understand how PAD affects gait patterns and to evaluate the effectiveness of different treatment methods. It was determined that conservative treatment may not be effective, and supervised exercise therapy and surgical intervention improve, but do not restore gait. In the course of developing and testing new treatment approaches, including ankle-foot orthoses and exoskeleton footwear, the data has consistently demonstrated gait dysfunction compared with healthy older individuals in controlled, appropriately powered, and carefully designed studies. These evaluations have collectively produced one of the largest datasets of biomechanics data from patients with PAD. The data reveals unique walking patterns in patients with PAD compared to older controls without PAD. Key differences include i) decreased peak push-off at the end of the stance phase and ii) decreased contributions in joint power during each phase of stance.

Embodiments herein determine the gait signatures of patients with PAD; meaningful measures from the laboratory based advanced biomechanics data are extracted and used to develop and train models to identify unique movement features and then test the model using novel data.

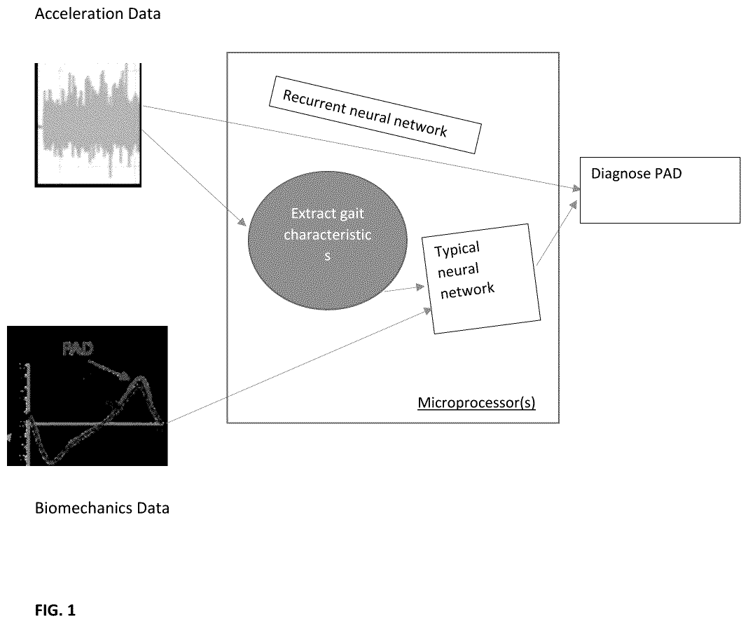

In an embodiment, biomechanics and/or acceleration data collected concurrently is used to train a model to more accurately classify PAD patients compared to the model trained directly from acceleration data alone (top in FIG. 1). For example, in an embodiment as shown in FIG. 1; acceleration data and/or biomechanics data may be fed to a system including one or more microprocessors configured to implement the training and characterization and diagnoses methodologies as described herein. Acceleration data provided to the system, either initial training data, or patient data for diagnosis, may be processed to extract gait characteristics as discussed herein; that gait characteristics data is then fed to a machine learning model, either to train the model in the case of the initial training data, or to analyze the data to determine one or more gait features for input to a trained model for a diagnosis in the case of the patient data. Additionally or alternatively, biomechanics data may also be fed to the machine learning model, either to train the model in the case of initial training data, or to analyze the data using a trained model for a diagnosis in the case of the patient data.

The present embodiments characterize gait in patients with PAD from biomechanics data and/or from wearable accelerometer data and/or ground reaction forces (GRF) data and/or kinematics data. Kinematics data may be collected or recorded using a digital camera, e.g., a high-speed digital camera motion capture system (e.g., 100 Hz; Cortex 5.1, Motion Analysis Corp., Rohnert Park, CA, USA) and ground reaction forces may be collected using force plates (e.g., 1000 Hz; AMTI, Watertown, MA, USA) or wearable sensors.

Software, such as Visual 3D software, may be used to process the collected data and to calculate the ground reaction forces in vertical, anterior-posterior, and medial-lateral directions, as well as ankle, knee, and hip joint angles, and joint angular velocities during the stance phase of walking. Joint torques and powers may calculated using inverse dynamics for the ankle, knee, and hip joints during the stance phase of walking. Inverse dynamics combines the kinematics and the ground reaction forces described. The joint torques and powers determine the lower extremity joint angles, muscular responses (torques), and contributions (powers) during walking. Al-Ramini et al. (Machine Learning-Based Peripheral Artery Disease Identification Using Laboratory-Based Gait Data, Sensors 2022, 22, 7432), which is incorporated by reference herein, discusses various numeric gait characteristics characterized by GRF and joint angles, torque sand powers useful in embodiments herein.

According to an embodiment, a method of diagnosing PAD in a patient includes first training a machine learning model implemented in a computer system including at least one processor and associated memory. The learning model may be trained with the gathered biomechanics data or gait characteristic data extracted from acceleration data for patients known to have PAD and patients that do not have PAD. For the case of the model trained by the gait characteristic data extracted from acceleration data, a human subject is then outfitted with one or more wearable sensor and asked to walk for a short period of time. Acceleration data related to the patient is gathered by the sensors as the patient walks. The acceleration data may be stored and later accessed by the processor, or may be directly supplied form the senor(s) to the processor after walking or in real-time as the patient walks. The processor receives the acceleration data for the patient and extracts gait characteristic data from the acceleration data. The extracted gait characteristic data may then be fed to the trained machine learning model to enable the model to identify one or more gait features for the specific patient. In some embodiments, the processor may also diagnose the specific patient as having PAD or not having PAD based on the one or more identified gait features, e.g. by way of digital output rendered on a display device or transferred to another device for display or rendering. For the case of the model trained by the biomechanics data, a human subject is asked to walk in a lab where a high-speed digital camera motion capture system can use to extract the biomechanics data. The biomechanics data may be stored and later accessed by the processor, or may be directly supplied form the senor(s) to the processor after walking or in real-time as the patient walks. The processor receives the biomechanics data for the patient which may then be fed to the trained machine learning model to enable the model to identify one or more gait features for the specific patient. In some embodiments, the processor may also diagnose the specific patient as having PAD or not having PAD based on the one or more identified gait features, e.g. by way of digital output rendered on a display device or transferred to another device for display or rendering.

The machine learning model may include any useful machine learning or artificial intelligence algorithm as would be apparent to one skilled in the art. Specific examples include a neural network algorithm, a nearest neighbor algorithm, a random forest algorithm, a support vector machine (SVM) algorithm and a Logit algorithm.

In an embodiment, the one or more gait features extracted from the acceleration measurements may include one or more of the following: step time asymmetry, step time variability, step time, stance time, stride time, and swing time. In an embodiment, the gait characteristic data may include one or more of the following: vertical acceleration, anterior acceleration, a number of steps, and a period of time between steps. In an embodiment, gait characteristic data may include biomechanics data such as braking impulse, braking peak, propulsive peak, propulsive impulse, and other forces derived from GRF data. In an embodiment, gait characteristic data may include joint torques and powers, such as hip, knee and/or ankle angles, torques and powers

Appendix A of U.S. Provisional Application No. 63/476,862, disclose additional details and embodiments of systems and methods for diagnosing PAD using trained machine learning or artificial intelligence algorithms, and is incorporated by reference herein.

### Data Gathering Example

Patients with peripheral artery disease were recruited from the vascular clinics at the University of Nebraska Medical Center and the Omaha VA Medical Center. Subjects were aged 50 years and older, had a stable blood pressure, lipid, and diabetes regimen for 6 weeks, and positive history of chronic claudication, exercise-limiting claudication per history and direct observation, and evidence of occlusive disease on ankle/brachial index testing and/or computerized tomographic angiography. Subjects were excluded if walking capacity was limited by conditions affecting the legs (joint/musculoskeletal, neurologic) and systemic (heart, lung disease) pathology. Patient comorbidities not impacting walking capacity such as obesity and diabetes will be included as factors in the training data. Healthy older individuals had the same inclusion/exclusion criteria except they had an ankle-brachial index above 0.90 and no history of claudication or exercise limitation as determined by a health history questionnaire. Patients without symptoms but with reduced blood flow (asymptomatic PAD) were not included.

Learning curves were used to evaluate classification accuracy. The available sample includes 200 baseline samples for patients with PAD, 75 healthy older individuals, and 206 follow-up patient visits (Table 1, data available includes 482 visits for patients with PAD and 75 visits for healthy older individuals), which is greater than the recommended number of samples needed to achieve accurate classification performance.

TABLE 1

| | | | Total | Trials/ | Strides/ | Outcomes |
|---|---|---|---|---|---|---|
| Dataset | N | Visits | Visits | visit | trial | (# features) |
| Exercise/ | 135 | 2 | 270 | 10 | 3-5 | Biomechanics |
| Surgery | | | | | | (45), walking |
| | | | | | | distances (2), |
| | | | | | | accelerometer |
| | | | | | | (8; home), |
| | | | | | | muscle |
| Endovascular | 20 | 4 | 80 | 10 | 3-5 | strength (16) |
| versus Open | | | | | | Biomechanics |
| Surgery | | | | | | (45), walking |
| | | | | | | distances (2), |
| | | | | | | muscle |
| | | | | | | activity (14) |
| Ankle Foot | 44 | 3 | 132 | 10 | 1 | Biomechanics |
| Orthosis | | | | | | (45), walking |
| | | | | | | distances(2), |
| | | | | | | accelerometer |
| | | | | | | (8; lab and |
| | | | | | | home), muscle |
| | | | | | | activity (14), |
| | | | | | | muscle |
| | | | | | | morphometrics |
| | | | | | | (4), muscle |
| | | | | | | oxygenation, |
| | | | | | | muscle |
| | | | | | | strength (16) |
| Cumulative | 75 | 1 | 75 | 10 | 3-5 | Biomechanics |
| Healthy Older | | | | | | (45), 6 minute |
| | | | | | | Controls |
| | | | | | | walk (2), |
| | | | | | | accelerometer |
| | | | | | | (8; lab), |
| | | | | | | muscle |
| | | | | | | morphometrics |
| | | | | | | (4), muscle |
| | | | | | | oxygenation |
| | | | | | | (12), muscle |
| | | | | | | strength (16) |

In a pilot study with 31 subjects from the dataset, a comparison was made of the accuracy of using all the biomechanics features to randomly selected sets of 40, and 33 features to distinguish patients with PAD from healthy older individuals. In each set, the dataset was randomly split into 75% training and 25% testing. An accuracy of 99% was achieved using all the features, 97% with 33 features and 95% with 40 features. This implies some features have a more significant impact on the model than others. This led to an understanding that a minimum set of biomechanics features can accurately characterize the PAD gait signature.

In an embodiment, to reduce the biomechanics features size; dimensionality reduction approaches may be used. This allows one to find fewer features that independently capture the main gait characteristics without losing significant information. In an embodiment, the dimensionality reduction may be performed using different methods: extracting features that represent the significant data characteristics (feature extraction) and selecting the most relevant features showing different gait signatures for patients with PAD (feature selection) from the original dataset.

In an embodiment, the minimum set of biomechanics features that capture the unique signatures in PAD patients is determined and these features are analyzed thoroughly to establish the “prior knowledge” for the machine learning algorithms to diagnose PAD using acceleration measurements.

Biomechanics features are specialized, expensive, and only provide a snapshot of gait. The present embodiments advantageously provide a new approach that leverages knowledge from biomechanics data to build models from acceleration data to aid PAD diagnosis, track disease progression, and monitor treatment impact.

In an embodiment, machine learning algorithms, such as RNN, SVM, etc., and a process of transfer of learning approach is used to transition the model from biomechanics to acceleration data. This advantageously leverages the knowledge (e.g., features, weights, etc.) from biomechanics data, improves accuracy, and reduces the training sample size for the newer models embodied herein (FIG. 1). To reduce the number of unknowns, biomechanics data and accelerometer measurements obtained from the same controlled experiment (Ankle Foot Orthosis Dataset) may be used.

To evaluate the accuracy of the PAD diagnostics models, well-established classification assessment methods such as the confusion matrix approach are used to report the percentages a model correctly predicts a subject has PAD-true positives (TP)—or not-true negatives (TN), and differentiate the instances the model falsely predicts that a subject has PAD, when the subject does not (false positives (FP)) or predicts presence of PAD when PAD is not present (false negatives (FN)). This is important as the impact of predicting an FN case is more severe than predicting an FP case. Finally, to avoid model overfitting and achieve better accuracy, a cross-validation, e.g., 10-folds of cross-validation, may be used, where in each iteration a portion of the data is randomly selected for modeling and the rest for testing.

In an embodiment, PAD-specific gait signatures are extracted from acceleration data. In some instances, the biomechanics-based signature may not be accurately represented with data from one accelerometer. Hence, in an embodiment, multiple sensors/accelerometers are used at varying locations on a patient's body.

### Data Sources and Preprocessing

The present embodiments include different ways to use acceleration measurements of a body part to diagnose a patient with PAD. The acceleration measurements may be obtained by double differentiating the body motion of the sacral position, obtained using a reflecting marker. Ultimately, the acceleration can be measured directly by a wearable accelerometer.

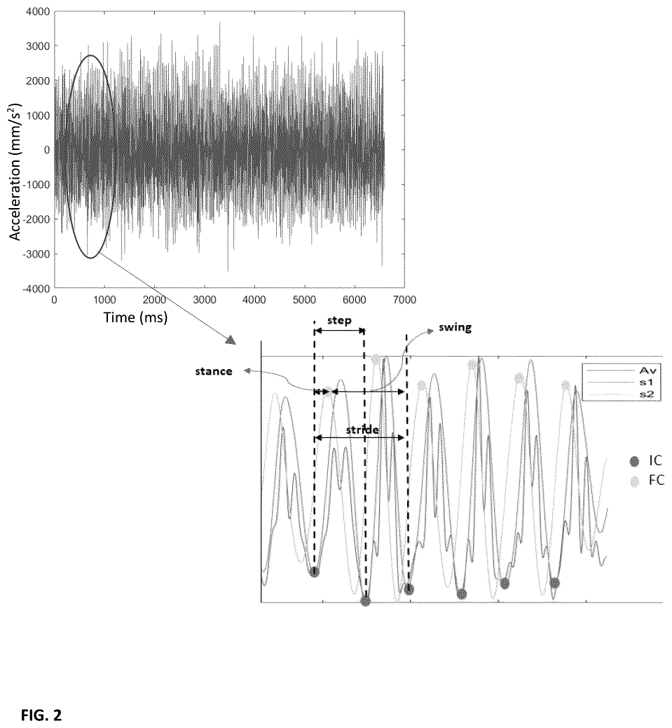

Next, the retrieval and data processing of the reflecting marker data to obtain the acceleration measurements and other features to train the machine learning models is explained. The dataset was collected in three dimensions (x, y, z) with a sample rate of 60 Hz. The x-axis denotes anterior-posterior, the y-axis represents mediolateral, and the z-axis represents vertical. The data is collected from 25 healthy individuals and 27 patients with PAD. The human subjects participated in flat walking treadmill trials that included many gait cycles. Two processing steps were used next. The first step (S1) is to derive acceleration from the sacral position marker data and the second step (S2) is to extract the gait characteristics from the derived acceleration measurements. FIG. 2 shows a graphic demonstration of gait cycles derived from the vertical dimension of acceleration obtained from the sacral position data (Av). S1 was derived from Gaussian CWT at a scale 10 of vertical acceleration and S2 was obtained from S1 differentiation. IC is S1 local minima and FC is S2 local maxima. Since we have sequential walking, we assume the first gait characteristics (like a step) as left (or right), and then alternate values.

### Step 1: Acceleration Derivation

The difference between each pair of successive sample values (rows) is utilized to calculate displacement.
Velocity is calculated by dividing displacement by the time interval between every two consecutive points (1/60 s).
The difference between each consecutive sample velocity (rows) is used to calculate instantaneous velocity.
Acceleration is calculated by dividing instantaneous velocity by the time gap between each pair of adjacent points (1/60 s).
Noises were removed using the most popular method, a fourth-order Butterworth filter with a 15 Hertz cutoff frequency (Hz).

### Step 2: Gait Characteristics Extraction

Identify the initial contact (IC, i.e., heel strike) and final contact (FC, i.e., toe-off) of a gait signal using the wavelets temporal approach as shown in FIG. 2. In FIG. 2 the following singles and points are shown:
Av is the original acceleration signal
S1, S2 are the wavelet-transformed signal and its derivative, respectively
IC and FC are the minima and maximum of S1 and S2, respectively.

### Extract the Following Gait Parameters:

step time: the interval between successive ICs
stance time: time between the first heel strike and toe-off
stride time: three ICs
swing time: the difference between stride and stance time.

### Calculate Three Types of Variabilities, for Each Gait Parameter as Follows:

general variability=σ(gait parameter), where a is the standard deviation
leg variability=√((variance_{left} and variance_{right})/2), where variance_{left} and variance_{right} represent the variance of the gait characteristic for the left leg and right leg, respectively.
leg asymmetry=|average_{left}−average_{right}|, where the average left and average right represent the mean of the gait characteristic for the left leg and right leg, and asymmetry is the absolute mean difference between the right leg and left leg.

In summary, motion data were collected at 60 Hz for a minute of flat walking from 27 PAD patients and 25 healthy controls. This data was processed to obtain 16 gait characteristics for each participant. In the next section, applying machine learning algorithms to this data to diagnose patients with PAD is discussed.

### Methods

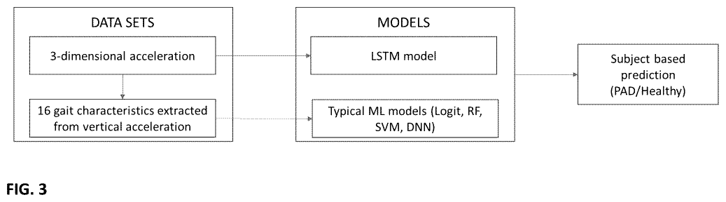

Embodiments discussed herein include two different models to discriminate between PAD patients and healthy controls. FIG. 3 is a flowchart showing two methods useful for PAD diagnostics: 1) using the raw acceleration directly and feeding it to a time-series machine learning model such as the LSTM model; and 2) utilizing the complexity of the 16 gait characteristics extracted from acceleration measurements to train typical machine learning models for PAD prediction. The first model utilizes the complexity of recurrent neural network (RNN), which deals with time series data such as Long short-term memory (LSTM), to allow using the raw acceleration measurements directly. In the second model, typical machine learning algorithms models such as logistic regression, random forest, support vector machine, and deep neural network are trained by 16 gait characteristics extracted from the raw acceleration measurements. Compared to the first model, the complexity in the second model is pushed toward the model input data rather than the model algorithm.

### LSTM Model Using Raw Acceleration Measurements

LSTM is an RNN with multiple layers of connected neurons. The LSTMs, like RNNs, include recurrent connections, which allow the state of the neuron from earlier activations in the preceding time step to be used as background for forming an output. To make a prediction, input data is propagated through the network. LSTM networks were explicitly designed to solve the RNNs issue with long-term dependencies. Because they have feedback connections, LSTMs differ from more traditional feedforward neural networks (the second model approach herein). This characteristic allows LSTMs to handle entire data sequences (like time series) without considering each data point in isolation. Instead, they can analyze current data by referring to preliminary data in the series.

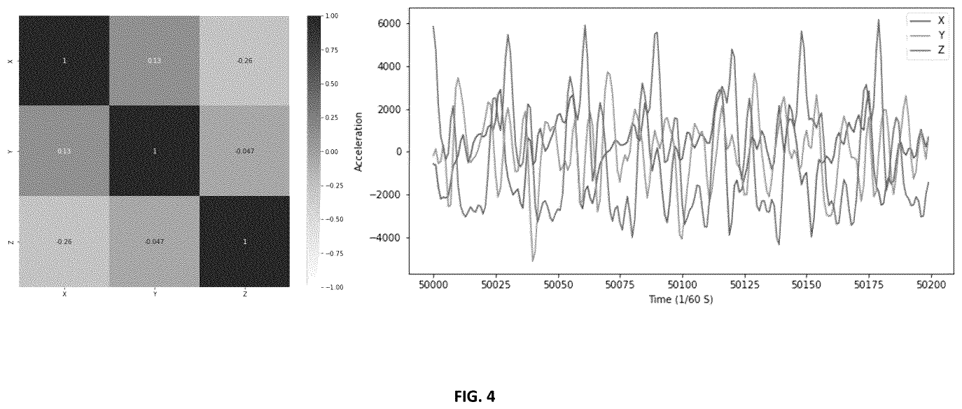

To train the LSTM model, the accelerometer data is divided into a training set with 16 PAD and 15 healthy subjects, a validation set with 4 PAD and 4 healthy subjects, and a test set with 7 PAD and 6 healthy subjects. Various numbers of PAD and healthy subjects in train, validation, and test sets were attempted before to reach the above combination that results in the highest accurate prediction. Moreover, different combinations of the row acceleration time-dependent signals x, y, and z were used as input data for the LSTM model. The goal is to find the minimum accelerometer set that enables faster and more accurate classifiers. This was driven by the correlation study findings in FIG. 4 that show a correlation between some of the accelerometer axis measurements. In FIG. 4, the left plot shows the correlation between the x, y, and z-axis of the acceleration data, and a sample of three-dimensional acceleration can be seen in the right plot.

To harness the full LSTM network's capability, the following hyperparameters were tuned: the number of steps (lookback size), maximum epochs, batch size, number of layers, number of neurons in each layer, activation functions, and optimizers. Table 2, below, depicts a LSTM model architecture along with its tuned hyperparameters. This architecture has activation functions before each hidden layer, two hidden layers, and a binary classification output activation function.

TABLE 2
The list of LSTM hyperparameters and the final
values obtained from the grid search:

| Hyper Parameter | Value |
|---|---|
| Num steps | 100 |
| Num features | 2 |
| Max epochs | 1 |
| Batch size | 4 |
| Optimizer | ‘adam’ |
| Layers | 3 layers (4, 4, 1) |
| Activation function | Tanh, Sigmoid |

### Standard Machine Learning Models Using the Extracted Gait Characteristics

In the second modeling approach, Logistic regression (Logit), Random Forest (RF), Support Vector Machines (SVM), and Deep Neural Networks (DNN) were used to predict PAD versus healthy subjects using the extracted gait characteristics. These algorithms have previously been employed in various classification tasks for diagnostics applications in the medical area. See, e.g., L. Ara, X. Luo, A. Sawchuk, and D. Rollins, “Automate the Peripheral Arterial Disease Prediction in Lower Extremity Arterial Doppler Study using Machine Learning and Neural Networks,” in Proceedings of the 10th ACM International Conference on Bioinformatics, Computational Biology and Health Informatics, New York, NY, USA, Sep. 2019, pp. 130-135. doi: 10.1145/3307339.3342180, and A. M. Flores, F. Demsas, N. J. Leeper, and E. G. Ross, “Leveraging Machine Learning and Artificial Intelligence to Improve Peripheral Artery Disease Detection, Treatment, and Outcomes,” Circ Res, vol. 128, no. 12, pp. 1833-1850, Jun. 2021, doi: 10.1161/CIRCRESAHA.121.318224.

Similar to the LSTM model approach, the data was divided into 19 healthy adults and 20 PAD patients for training and 6 healthy individuals, and 7 PAD patients for testing. To impute missing values, each feature was grouped by participants and assigned the mean of the feature to the missing value.

Logit model: The model summary and corresponding p-value of variables were utilized to pick out the most crucial ones. This pre-processing step resulted in dropping 4 variables: ‘StepTime’, ‘StanceTime’, ‘StrideTime’, and ‘SwingTime’).

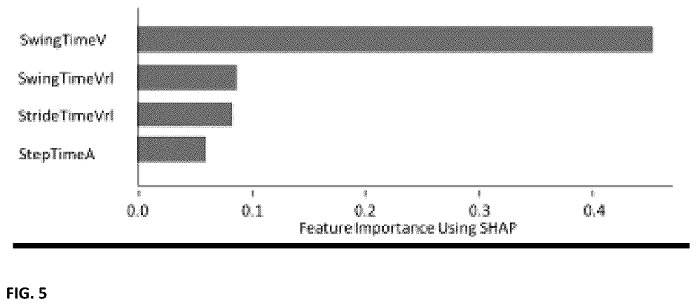

RF model: The hyperparameter parameters listed in Table 3 are tuned using the Bayesian hyperparameter tuning algorithm with 5-fold cross-validation. The less significant features were found and removed based on their SHAP values. The SHAP value has been presented as a way to quantify feature importance since the importance value it assigns to each feature reflects the role it plays in model prediction. Finally, the top 4 features (presented in FIG. 5), were used to train the final RF model.

TABLE 3
Hyperparameters used to tune the RF model,
their range, and the result of tuning:

| Hyperparameter | Range | Selected |
|---|---|---|
| Criterion | ‘entropy’, ‘gini’ | ‘entropy’ |
| max_depth | range (0, 20) | 1 |
| max_features | ‘auto’, ‘sqrt’, ‘log2’, none | ‘log2’ |
| min_samples_leaf | uniform(0, 0.3) | 0.1585 |
| min_samples_split | uniform(0, 1) | 0.106 |
| n_estimators | range(0, 100) | 78 |

### SVM

A similar approach to the RF model was used to find the Hyperparameters for the SVM model. The final parameters are presented in Table 4.

TABLE 4
Hyperparameters used to tune the SVM model,
their range, and the result of tuning:

| Hyperparameter | Range | Selected |
|---|---|---|
| C | range(0, 100) | 65 |
| kernel | ‘linear’, ‘poly’, ‘rbf’, ‘sigmoid’ | ‘poly’ |
| degree | 1, 2, 3, 4, 5 | 4 |
| probability | probability | 0 |

### DNN

The Bayesian hyperparameter tuning was used to tune the DNN hyperparameters such as the number of hidden layers, the number of neurons in each hidden layer, activation functions, optimizers, and maximum epochs. Based on our previous findings we also dropped the following features: ‘StepTime’, ‘StanceTime’, ‘StrideTime’, and ‘SwingTime’). The final list of hyperparameters of the DNN model is shown in Table 5.

TABLE 5
Hyperparameters used to tune the DNN model,
their range, and the result of tuning:

| Hyperparameter | Range | Selected |
|---|---|---|
| layers | range(2, 5) | 4 |
| neurons | arange(2, 256, 4) | 64, 256, 64, 1 |
| dropout | arange(.20, .75, 0.025) | 0.7 |
| batch size | arange(4, 128, 8) | 44 |
| activation function | ‘relu’, ‘tanh’, ‘sigmoid’ | ‘sigmoid’ |
| optimizer | ‘adadelta’, ‘adam’, ‘rmsprop’, ‘adagrad’, ‘sgd’ | ‘adam’ |

To evaluate the different models herein, the typical machine learning metrics such as accuracy, precision, recall, and F1 score were used.

### Results

As shown in Table 6, the LSTM model achieved the best accuracy of 92% when it is trained using the y and z (mediolateral, and vertical) raw acceleration measurements. Moreover, Table 6 shows that the x-axis acceleration measurement has the highest accuracy if only one acceleration measurement is available. This is somewhat expected from the correlation analysis in FIG. 4 where the x-axis acceleration has the highest correlation with the other axis measurements.

TABLE 6
All possible combinations of 3-axis time-series data fed to
the LSTM model and the metrics obtained from predictions:

| Combination | Accuracy (%) | Precision (%) | Recall (%) | F1 (%) |
|---|---|---|---|---|
| X, Y, Z | 77 | 100 | 70 | 82 |
| X, Y | 62 | 71 | 62 | 66 |
| X, Z | 85 | 100 | 77 | 87 |
| Y, Z | 92 | 100 | 87 | 93 |
| X | 77 | 86 | 75 | 80 |
| Y | 54 | 100 | 54 | 70 |
| Z | 69 | 100 | 64 | 78 |

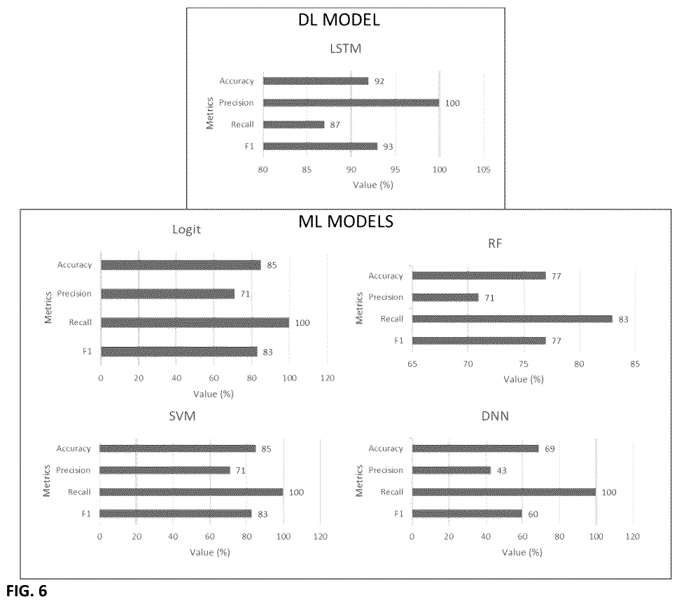

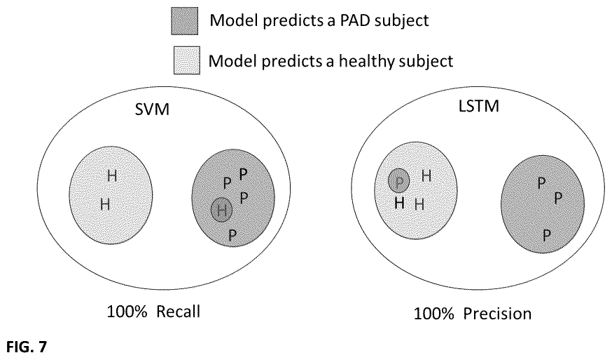

FIG. 6 shows a schematic comparison of the performance of ML and LSTM models. Accuracy, Precision, Recall, and F1 score are the metrics used for comparison. In FIG. 6, the performance of the different machine learning models was evaluated to predict patients with PAD using the gait characteristics extracted from vertical acceleration measurements. For comparison purposes, in FIG. 6 the best performance of the LSTM model is shown. FIG. 6 shows that the LSTM model still has the highest accuracy of 92% compared to 85% using Logit models or the SVM model. However, FIG. 6 shows that the SVM and Logit models have a 100% recall rate. This indicates while these models have falsely declared some healthy subjects to have PAD, they have identified all the PAD patients. Whereas, while each PAD prediction for the LSTM model is valid (100% precision), the model has not predicted all the PAD patients (FIG. 7). Thus, in real field implementation, while they are less accurate, one may prefer to use the SVM or the Logit models over the LSTM model. Specifically, as the final diagnostics model is to be used at home, away from a clinical setting, it is important to flag any potential PAD case for a medical follow-up rather than missing a real case.

Another implementation is to use the LSTM concurrent with the SVM (for example) model. In this implementation, any PAD cases flagged by the LSTM will be valid (LSTM has 100% precision) and any Healthy cases flagged by the SVM model will be valid (SVM has 100% recall). This combined model implementation will produce results that exceed the performance of either individual model. Actually, as shown in Table 7, the only reason this combined model doesn't produce perfect results is when the LSTM predicts Healthy and the SVM predicts a PAD. In this case, as shown in the blue circles in FIG. 7, the final prediction has a chance to be wrong. FIG. 7 is a schematic explaining the advantages of the SVM and LSTM models. The LSTM model has the advantage of predicting 100% valid patients with PAD, whereas the SVM model has the advantage of predicting 100% valid Healthy subjects. However, the schematic shows that LSTM might miss real PAD cases, and the SVM, which captures all the PAD patients, might declare wrongly a healthy subject to be a PAD patient (the blue circles).

TABLE 7
The LSTM and the SVM models simple logic to
produce a better performance combined model:

| The actual | LSTM | SVM | Final |
|---|---|---|---|
| Case | prediction | Prediction | prediction |
| PAD | PAD | X | PAD |
| PAD | Healthy | PAD | ? |
| Healthy | Healthy | PAD | ? |
| Healthy | X | Healthy | Healthy |

This modeling strategy should encourage the extraction of acceleration and gait features using wearable sensors that could be worn in real-world settings and used outside the lab to diagnose and detect possible PAD patients. Thus, the present embodiment advantageously enable continuously tracking people's activity and physical health behaviors. PAD is expensive for people, societies, and governments. These new models enable in-home diagnosis of worsening PAD symptoms, managing chronic PAD, and anticipating when severe adverse health events may occur. They would also alert physicians to the possible existence of PAD in general practices. Finally, researchers can utilize these machine learning embodiments to identify PAD or other diseases by utilizing the same preprocessing of acceleration time-series data, gait feature extraction, model architecture, and performance metrics outlined herein.

The present embodiments provide:
1) Validation of a gait signature in PAD. In contrast, previous reports exploring gait signatures of 26 different chronic conditions did not include PAD. Gait investigations of PAD have detailed limitations compared to controls and described the relationship of those limitations to clinical measures but have not developed predictive or assessment models using gait parameters.
2) Use of machine learning on a new PAD data source; gait data. In contrast, prior machine learning for patients with PAD previously used blood samples, Doppler data, clinical records and symptom surveys, interviews, and walking distances. Gait data as used herein advantageously presents an opportunity to identify PAD prior to symptom onset and, in an embodiment, may be used in conjunction with sources mentioned above. Gait signatures can advantageously be captured from wearable devices outside of the clinical setting.
3) A new clinical approach. With accurate models for predicting PAD outcomes, patients can be monitored from afar. The present embodiments can be configured to alert physicians for problematic or significant changes in movement patterns. The models could also may be used in conjunction with the standard treatment visits to provide quantitative context to functional problems described by patients. This would help physicians gauge changes from one visit to the next, know whether a treatment is effective, or indicate an intervention is needed. Being able to monitor in a natural environment and at multiple time points remotely also provides a more representative picture, rather than the “snapshot” of a clinic visit.

While the initial embodiments are directed to patients with PAD, the embodiments herein can be expanded or modified to other chronic conditions affecting gait which would advantageously transform the clinical approach to chronic disease management.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A computer-implemented method of diagnosing peripheral artery disease (PAD) in a patient, the method comprising:
training a machine learning model with gait characteristic data, extracted from acceleration data for patients known to have PAD and patients that do not have PAD;
receiving acceleration data related to a specific patient;
extracting gait characteristic data from the acceleration data related to the specific patient;
feeding the extracted gait characteristic data to the trained machine learning model to identify one or more gait features for the specific patient; and
diagnosing the specific patient as having PAD or not having PAD based on the one or more identified gait features.

2. The method of claim 1, wherein the machine learning model comprises one of a neural network algorithm, a random forest algorithm, a support vector machine (SVM) algorithm and a Logit algorithm.

3. The method of claim 1, wherein the acceleration data related to the specific patient is obtained from one or more sensors worn by the specific patient.

4. The method of claim 1, wherein the one or more gait features include one or more of step time asymmetry, step time variability, step time, stance time, stride time, and swing time.

5. The method of claim 1, wherein the gait characteristic data includes one or more of vertical acceleration data, anterior acceleration data, a number of steps, and a period of time between steps.

6. The method of claim 1, wherein the gait characteristics data includes biomechanics data including one or more of braking impulse, braking peak, propulsive peak, propulsive impulse, other forces derived from ground reaction forces (GRF) data, and joint torques and powers, including hip, knee and/or ankle angles, torques and powers.

7. The method of claim 1, wherein the step of feeding further includes feeding biometric data of the specific patient to the trained machine learning model.

8. A system for diagnosing peripheral artery disease (PAD) in a patient, the system comprising:
a processor and a memory storing instructions, which when executed by the processor causes the processor to access or execute a trained a machine learning model, the trained machine learning model having been trained with biometric data and gait characteristic data extracted from acceleration data for patients known to have PAD and patients that do not have PAD; and
one or more sensors configured to be worn by a specific patient;
wherein the processor is further configured to:
receive acceleration data related to the specific patient generated by the one or more sensors;
extract gait characteristic data from the acceleration data related to the specific patient;
feed the extracted gait characteristic data to the trained machine learning model to identify one or more gait features for the specific patient; and
diagnose the specific patient as having PAD or not having PAD based on the one or more identified gait features.

9. The system of claim 8, wherein code and data associated with the trained machine learning model is stored in the memory.

10. The system of claim 8, wherein the machine learning model comprises one of a neural network algorithm, a random forest algorithm, a support vector machine (SVM) algorithm and a Logit algorithm.

11. The system of claim 8, wherein the one or more gait features include one or more of step time asymmetry, step time variability, step time, stance time, stride time, and swing time.

12. The system of claim 8, wherein the gait characteristic data includes one or more of vertical acceleration, anterior acceleration, a number of steps, a period of time between steps, braking impulse, braking peak, propulsive peak, propulsive impulse, other forces derived from ground reaction forces (GRF) data, and joint torques and powers, including hip, knee and/or ankle angles, torques and powers.

13. The system of claim 8, wherein the instructions to feed the extracted gait characteristic data to the trained machine learning mode further include instructions to feed biometric data of the specific patient to the trained machine learning model.

14. A computer-implemented method of diagnosing peripheral artery disease (PAD) in a patient, the method comprising:
training a machine learning model with acceleration or accelerometer data for patients known to have PAD and patients that do not have PAD;
receiving acceleration data related to a specific patient;
feeding the acceleration data to the trained machine learning model to identify one or more gait features for the specific patient; and
diagnosing the specific patient as having PAD or not having PAD based on the one or more identified gait features.

15. The computer-implemented method of claim 14, wherein the machine learning model comprises a recurrent neural network or a long short-term memory (LSTM) model.

16. A non-transitory computer-readable medium storing instructions, which when executed by one or more processors, cause the one or more processors to implement a method of diagnosing peripheral artery disease (PAD) in a patient, the method comprising:
training a machine learning model with gait characteristic data extracted from acceleration data for patients known to have PAD and patients that do not have PAD;
receiving acceleration data related to a specific patient;
extracting gait characteristic data from the acceleration data related to the specific patient;
feeding the extracted gait characteristic data to the trained machine learning model to identify one or more gait features for the specific patient; and
diagnosing the specific patient as having PAD or not having PAD based on the one or more identified gait features.

17. The computer-readable medium of claim 16, wherein the acceleration data related to the specific patient is obtained from one or more sensors worn by the specific patient.

18. The computer-readable medium of claim 16, wherein the one or more gait features include one or more of step time asymmetry, step time variability, step time, stance time, stride time, and swing time.

19. The computer-readable medium of claim 16, wherein the gait characteristic data includes one or more of vertical acceleration data, anterior acceleration data, a number of steps, and a period of time between steps.

20. The computer-readable medium of claim 16, wherein the gait characteristics data includes biomechanics data including one or more of braking impulse, braking peak, propulsive peak, propulsive impulse, other forces derived from ground reaction forces (GRF) data, and joint torques and powers, including hip, knee and/or ankle angles, torques and powers.
